(19) United States
(12) Patent Application Publication
(10) Pub. No.: US 2026/0226139 A1
(43) Pub. Date: Aug. 6, 2026

(54) CELL CULTURE METHODS

(71) Applicant: Amgen Inc., Thousand Oaks, CA (US)

(72) Inventors: Madiha KHURSHID, Simi Valley, CA (US); Neil SOICE, Simi Valley, CA (US); Nitya Mariam JACOB, Oak Park, CA (US); Jessica A. PEDROSO, Ashland, MA (US); Sidney PEHRSON, Fuquay Varina, NC (US)

(73) Assignee: Amgen Inc., Thousand Oaks, CA (US)

(21) Appl. No.: 19/152,699

(22) PCT Filed: Jan. 25, 2024
(86) PCT No.: PCT/US2024/012849

§ 371 Date: Jul. 31, 2025

Related U.S. Application Data
(60) Provisional application No. 63/443,190, filed on Feb. 3, 2023.

(51) Int. Cl. C07K 16/00

(52) U.S. Cl. CPC C07K 16/00; C07K 2317/14

(57) ABSTRACT

The disclosure provides non-steady state continuous perfusion cell culture methods for protein production in bioreactors. Cells are cultured in non-steady state after entering production phase such that the viability declines over time. Production of protein products using these cell culture methods is increased.

### CROSS-REFERENCE TO RELATED APPLICATIONS

This application claims priority to Provisional Application No. 63/443,190, filed Feb. 3, 2023, which is incorporated herein by reference in its entirety.

### FIELD

The disclosure provides non-steady state continuous perfusion cell culture methods for protein production in bioreactors. Cells are cultured in non-steady state after entering production phase such that the viability declines over time. Production of protein products using these cell culture methods is increased.

### BACKGROUND

A variety of cell culture methods are used to produce recombinant biopharmaceutical proteins from bioreactors. Protein product yield and process time associated with the methods remain areas for development in the bioprocessing field. Variations in protein product manufacturing that increase productivity, improve consistency of product, reduce cost of starting materials or processing time, or reduce equipment costs can have substantial economic benefits.

Bioprocessing can be carried out in batches (most commonly fed-batch processing) or in continuous perfusion systems. Mammalian cells such as Chinese hamster ovary (CHO) cells are frequently used for bioprocessing. In batch systems, protein products are harvested at the end of the culture run. In perfusion systems, cell cultures are regularly fed with fresh media and protein product-containing media fluid is harvested continually throughout a cell culture run. Perfusion systems use filtration-based retention devices to capture protein products and/or cells from a run by, for example, tangential flow filtration, recirculating tangential flow filtration or alternating tangential flow filtration.

In a steady state perfusion system, the culture cell density is kept essentially constant by removing excess cells with the harvest fluid and the cells continually grow. In contrast, in a non-steady state perfusion system, cells are allowed to grow in the growth phase up to a user-defined maximum after which cell growth slows down and eventually stops to favor protein production.

The first steps of bioprocessing involve a series of scale-up and expansion phases designed to generate sufficient cell mass for inoculation of a production bioreactor. The cell culture process is initiated by thawing a vial from a working cell bank (WCB) and expanding the culture by using, for example, a series of shake flasks, culture bags, and/or expansion seed bioreactors (e.g., N−3, N−2, wherein the number indicates how many steps antecedent a bioreactor is from N, the final, or production, bioreactor), or the like. Following growth in the seed culture bioreactors, the culture is transferred to an N−1 bioreactor, which can be, for example, a perfusion bioreactor. In an N−1 perfusion bioreactor, the culture is perfused with fresh medium in order to generate sufficient cell densities for inoculation of the final cultivation step, the production bioreactor (N). The (N) production bioreactor is operated to maximize the efficient production of the protein product.

There remains a need in the art for increasing the productivity of perfusion systems for bioprocessing.

### SUMMARY

The disclosure provides methods of producing a protein product in a bioreactor in continuous perfusion mode. The method comprises a growth phase followed by a production phase which is not operated at steady state cell culture conditions. The growth phase comprises steps including, but not limited to, (a) inoculating cells expressing the protein product and liquid media into a bioreactor at a high cell density, and (b) growing the cells at a set temperature and progressively higher permeate rates to a first biomass setpoint; and the non-steady state production phase comprises steps including, but not limited to, (c) shifting to a lower temperature or a lower permeate rate when the first biomass setpoint is reached to begin to transition the culture towards protein production, (d) growing the cells at a set temperature and permeate rate up to a second higher biomass setpoint that promotes non-steady state cell culture and high productivity, (e) culturing the cells at the stated culture conditions in (d), such that the viability decreases over time, and (f) collecting the protein product from a harvest stream during the production phase.

The variable cell density (VCD) can decline over time and/or the packed cell volume (PCV) can increase over time.

One or more cell bleeds can be performed during the production phase so that the culture does not exceed a viability maximum. A bleed can occur once the cells increase past the second biomass setpoint. In that instance, the bleed rate in step (e) decreases or falls to zero when the culture is in non-steady state. A manual non-zero constant bleed can be employed in a non-steady state.

The high cell density of (a) can be about 0.2 million cells/mL to about 5 million cells/mL, about 1 million cells/mL to about 5 million cells/mL, or about 1 million cells/mL, about 2 million cells/mL or about 4 million cells/mL.

The first biomass setpoint can be about 50 million cells/mL to about 100 million cells/mL. The second biomass setpoint can be about 100 million cells/mL to about 150 million cells/mL.

The permeate rate of (b) can be 0 to about 4.1 working volumes/day. In step (b), the permeate rate increases as the biomass increases to support the growth of the cells. The permeate rate of (d) can be about 1.0 to about 4.2 working volumes/day.

The VCD maximum of (d) can be about 130 million to about 140 million cells/mL. The VCD can decline in the production phase to about 10 million cells/mL to about 120 million cells/mL.

The set temperature of (b) can be about 35.5 to about 36.5° C., or for example about 36° C.

The set temperature shift of (c) and (d) can be a lowering of the temperature to about 32.5-35.5° C.

The permeate rate of (c) and (d) to induce a shift can be a lowering to about 1.0 to about 2.5 working volumes/day.

The cell bleed in the growth phase can be from 0% to about 40%. The cell bleed in the growth phase can be from 0% to about 3%. The cell bleed in the growth phase can be about 1.5%.

The cell viability in the growth phase can be about 90% to about 99%. The cell viability in the growth phase can be about 97%. In step (e), the viability decreases over time to a viability of about 30% to about 80%, about 30 to about 75%, about 35 to about 70%, about 35 to about 60%, about 35 to about 50%, or about 80%, about 75%, about 70%, about 65%, about 60%, about 55%, about 50%, about 45% or about 40%.

The PCV in the growth phase can increase to a value of from about 2% to about 24%. The PCV in the production phase can be about 25% to about 50% or about 40% to about 50%.

The duration of the growth phase can be about four to about 12 days. The duration of the production phase can be about nine to about forty-one days. The duration of the production phase can be about ten days.

The methods can further include subjecting the harvested protein product to downstream steps of capture chromatography, viral inactivation and/or polishing steps.

The cells in the methods can be mammalian cells. The mammalian cells can be Chinese hamster ovary (CHO) cells.

The protein product can be, for example, an antibody product. The protein product can be a bispecific antibody.

### BRIEF DESCRIPTION OF THE DRAWINGS

FIG. 1 shows daily trends over the course of a CM run for a first antigen-binding protein.

FIG. 2 shows daily trends over the course of a CM run for a second antigen-binding protein.

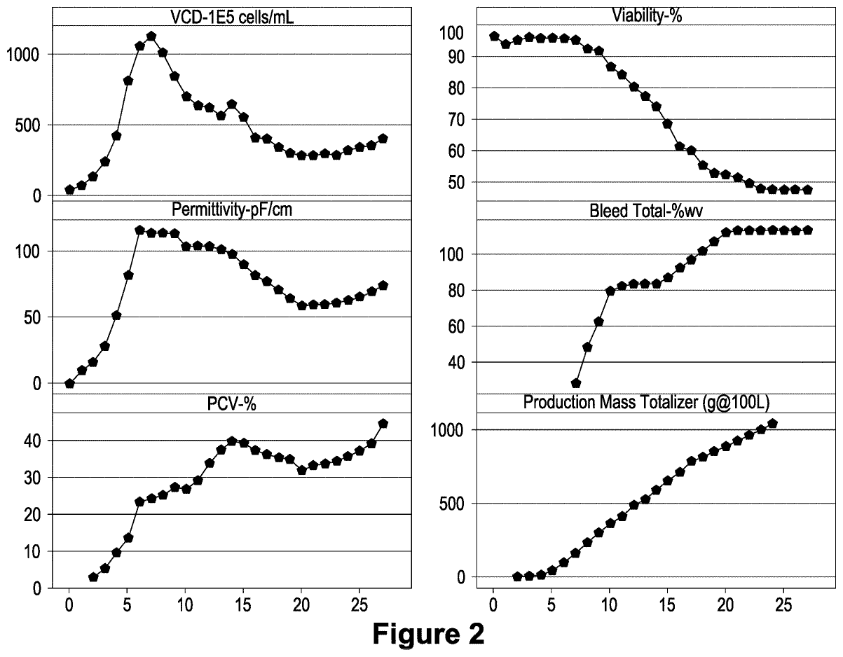

FIG. 3 shows daily trends over the course of a CM run for a third antigen-binding protein.

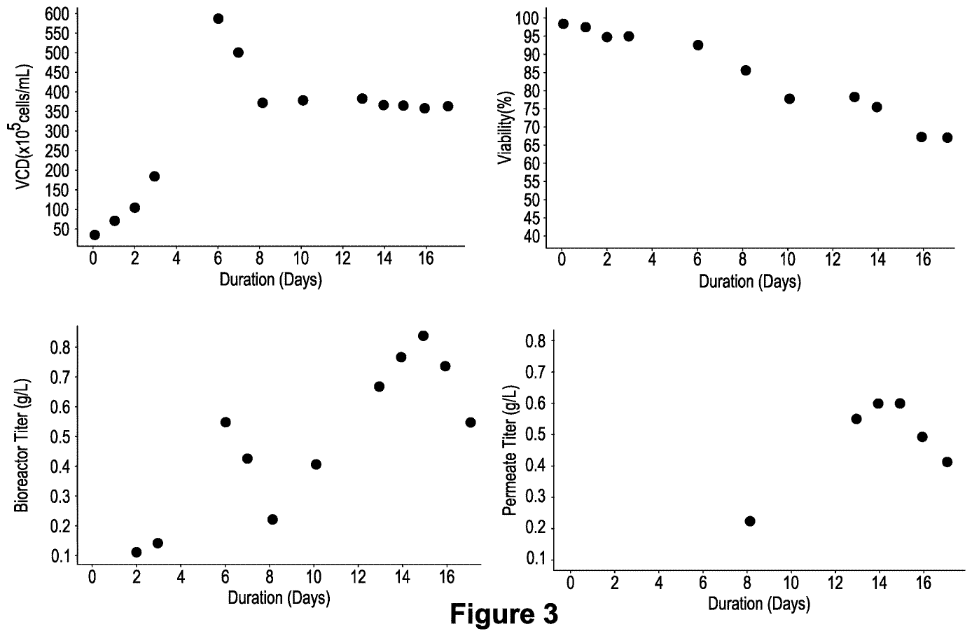

### DETAILED DESCRIPTION

Provided herein is a non-steady state continuous perfusion culture process for biologics manufacturing. Unlike traditional extended continuous perfusion technologies, the cell culture is not operated at steady state after reaching a user defined maximum peak growth. The process increases productivity since the cells are maintained in a production phase after cell growth plateaus and reduces culture duration, which improves productivity compared to a typical perfusion culture process. The production phase of the culture begins at the transition from the growth phase. The culture conditions are modified to favor product production over exponential cell growth. The viable cell density and viability are forced to decline due to change in culture conditions shifting the cells from a growth phase to a production phase. Once the set maximum is reached, the cell culture is shifted to production phase. This shift can be effected through a temperature shift or other means. Optionally there may be a brief biomass intensification at the start of the perfusion phase, with an optional cell bleed to maintain the biomass setpoint, but following that period of intensification, the biomass is allowed to decline well past the setpoint, resulting in no net cell growth. Bleeds can be carried out to ensure that the cell density does not exceed a maximum viable cell density (VCD). Where a bleed is used, as cell growth decreases bleed decreases as well. In the non-steady part of the production phase, the cell diameter and biovolume, as measured by, for example, Packed Cell Volume (PCV), increase and the biomass declines, since there is no net cell growth. It was found that under these conditions the cell diameter and biovolume, as measured by Packed Cell Volume (PCV), increased, indicating the cells stayed in a production phase.

Operating production bioreactors at non-steady state cell culture according to the disclosure surprisingly results in higher productivity (protein production) than operating at steady-state cell culture. This reduces the culture duration, and consequently increases bioreactor utilization and improves process economics. A production (N) bioreactor run herein can be carried out for an extended, flexible period, e.g., 15-35 days. The harvest yield increases in culture due to a reduction in bleed requirements. For example, a traditional steady state culture removes about 10% of the cells continuously over a 20-day culture period. By operating the culture in a non-steady state, the bleed can be cut in half or more. Thus, non-steady state operation according to the disclosure results in an economically favorable process. Protein product quality is similar across lots of non-steady state continuous manufacturing (CM) demonstrating the process is in a state of control, as required by government regulations.

As used herein, the term “viable cell density” or “VCD” refers to the number of live cells present in a given volume of medium (e.g., cells/mL) under a given set of experimental conditions. In methods herein, in a growth phase prior to a production phase the culture cell mass is grown to a maximum setpoint of a VCD of about 60×10⁶ cells/ml to about 143×10⁶ cells/ml, then the production phase is initiated in continuous perfusion mode, and the VCD is allowed to decline throughout the remainder of the culture. In the production phase, the VCD can decline at least 10%, at least 15%, at least 20% or at least 25% from the maximum setpoint. In the production phase, the VCD can decline at least 2%/day, at least 3%/day, at least 4%/day, or at least 5%/day. The methods can further comprise performing an initial bleed during the production phase to prevent the VCD from exceeding the maximum setpoint. A second maximum VCD setpoint can be used during the decline to prevent clogging of filtration apparatus.

As used herein, the “growth phase” of a cell culture refers to the phase during which the viable cell density at any time point is higher than at any previous time point. Cells can be in the growth phase for at least 4 days.

As used herein, the “production phase” of a cell culture refers to the phase during which the cells produce significant amounts of protein, which accumulates for future processing. For the production phase, the perfusion culture can continuously run for at least 7 days; for at least 14, 15, 16, 17, 18, 19, 20, 21, 22, 23, 24, 25, 26, 27 or 28 days; or for at least 35 days.

Permittivity (pF/cm) is a measure of the electric polarizability of a bioelectric. A material such as an (outer) cell membrane of a (living) cell with a high permittivity polarizes more in response to an applied electric material than a material with low permittivity. The electric displacement field D resulting from an applied electric field E is D=eE. More generally, the permittivity is a thermodynamic function of state. It can depend on the frequency, magnitude, and direction of the applied field. The SI unit for permittivity is farad per meter (F/M). Permittivity is measured with a permittivity probe (e.g., Hamilton Bonaduz AG, Switzerland). It can be measured in line or manually offline. Permittivity increases in the growth phase from about 4 pF/cm to a permittivity maximum of 60-115 pF/cm. The permittivity decreases in the production phase from the permittivity maximum, due to the non-steady state cell culture operation.

Permeate rate (wv/day) is a measure of the volume of media that is passed through and removed from the bioreactor system per day. Typically, this is measured in terms relative to the volume of the bioreactor, such as “bioreactor volumes” (bv) or “working volumes” (wv) per day. The permeate rate can be from about 1.0 to about 4.2 wv/day. During the production phase, the permeate rate is about 2.0.

Biomass-specific permeate rate (pF/cm·day) is derived from the permeate rate (1/day) divided by the permittivity (pF/cm). It is the fresh feed rate relative to the biomass in culture. Biomass-specific permeate rate, along with temperature, are process parameters that can be used to define the process design space as steady-state or non-steady state. This calculated rate is used to compare the process inputs across runs of a single cell line, since it combines two process parameters into one. Thus, by observing the temperature and Biomass-specific permeate rate at the biomass maximum, the process design space can be defined. If a run of the same cell line and product is repeated at the same temperature and biomass-specific permeate rate at the biomass maximum (relative ratio of perfusion rate and biomass maximum), similar results can be expected in terms of protein production and rate of biomass decrease (cell culture performance indicators).

Packed cell volume (PCV)—the biovolume of cells (sometimes referred to as cell mass or biomass herein) of the culture relative to the total volume of culture, expressed as a percentage. The PCV can increase as a result of an increase in number of cells, cell diameter in the same number of cells, or a combination. When the cell bleed is zero in continuous cultures (indicating no cell growth) and PCV continues to increase, it indicates that the cell diameter is increasing.

A “biomass capacitance probe” refers to a probe that can measure viable cell density, among other capabilities. A biomass capacitance probe uses capacitance to measure the total viable cells in a culture. Viable cells act as capacitors in an alternating electric field. The biomass capacitance probe can measure the charge from these cells and report it.

### Steady State and Non-Steady State

The CM culture design space consists of several process parameters which can result in conditions that favor cell growth over protein production (steady state CM) or conditions that favor protein production over cell growth (non-steady state CM). In steady state culture, all culture setpoints and outputs remain the same for the culture period. This is only possible if the cells are growing at a rate (cell growth rate) that maintains VCD, viability, PCV, and permittivity, but this prioritizes cell resources away from protein production towards cell growth. Typically in the art of continuous manufacturing, the VCD, viability and often the protein production rate are constant throughout the production period of culture. The cell growth is equal to the cell removal rate and the cells are both growing and producing protein.

However, in non-steady state culture, the cells grow slowly or stop growing altogether in favor of producing protein. This causes the cells to decrease in number and viability (due to some cell death) and expand in size, increasing cell diameter, to accommodate the increase in protein production. Thus, a non-steady state CM process results in a higher protein production rate and is therefore more economically attractive than traditional steady state processes.

There are degrees of magnitude in non-steady state operation. The performance indicators are measures of cell number, size and viability (i.e., VCD, viability, cell diameter, PCV, and permittivity) as well as protein production rate. The process parameters to tailor the magnitude of the impact on performance indicators are temperature and permeate rate relative to the biomass in culture (biomass-specific permeate rate). The process parameters can be modified to meet the goals for performance indicators. ATF filter fouling due to high biomass, cell debris, culture viscosity or high permeate rates puts a limit on the biomass maximum and permeate rate. In addition, a low temperature and permeate rate with a high biomass may arrest cell growth too quickly, which could end the culture before sufficient protein is produced. Thus, the biomass maximum (Permittivity Target), temperature and permeate rate are optimized in the process design space to meet the protein production goals, while taking process robustness into account.

In steady state operation, the permittivity/biomass is maintained at one single target level in the production phase. In non-steady state operation as described herein, the permittivity/biomass is maintained at or below a maximum in both parts of the production phase, a higher maximum in the biomass intensification phase and a second lower maximum in the remainder of the run. In non-steady state, the biomass needs to be maintained below a maximum to decrease the likelihood of fouling of the ATFs. The two-step maxima method (biomass intensification then lower maximum permittivity) mediates the risk of ATF fouling/failure with the advantages of greater productivity from the higher Permittivity Target.

### Biomass Intensification Phase

This phase refers to the increase in biomass up to a setpoint for a short period of time, prior to allowing the biomass to decrease. This intensification phase results in higher protein production in the remainder of the run. The higher the setpoint of the initial biomass phase, the more cells there are available for protein production in the remainder of the run, which leads to greater process productivity. This phase is optional in non-steady state CM. The biomass intensification phase is similar to the growth phase and is typically designed to take place immediately following the growth phase. The difference is that the biomass intensification phase has a lower (production phase) temperature setpoint and a different target permittivity, which is higher than the growth phase target permittivity.

### Cell Culture

Cell culture methods herein are carried out in production bioreactors in continuous perfusion mode, typically using alternating tangential flow filtration technology, in non-steady state operation. Cell culture refers to a liquid culture medium containing a plurality of cells that are maintained or proliferated under a controlled set of physical conditions.

Mammalian cells, such as CHO cells, can be cultured in small scale cultures, such as for example, in 100 ml containers having about 30 ml of media, 250 ml containers having about 35 to about 70 ml of media, or 500 ml containers having about 100 to about 200 ml of media. Alternatively, the cultures can be large scale such as, for example, 1000 ml containers having about 140 to about 300 ml of media, 3000 ml containers having about 500 ml to about 2200 ml of media, 50 L containers having about 4 L to about 30 L of media, and 200 L containers having about 50 L to about 135 L of media. Large scale cell cultures, such as for clinical manufacturing of protein therapeutics, are typically maintained for days, or even weeks, while the cells produce the desired protein(s).

The term “bioreactor” means any vessel useful for the growth of a cell culture, such as fluidized bed bioreactors, hollow fiber bioreactors, roller bottles, shake flasks, or stirred tank bioreactors. A bioreactor can be of any size so long as it is useful for the culturing of cells; typically, a bioreactor is sized appropriate to the volume of cell culture being grown inside of it. Typically, a bioreactor will be at least 1 liter and may be 2, 5, 10, 50, 100, 200, 250, 500, 1,000, 1500, 2000, 2,500, or 5,000 liters or more, or any volume in between. A bioreactor may be 8,000, 10,000, 12,000, 18,000, 25,000 liters or more, or any volume in between. The internal conditions of the bioreactor, including, but not limited to, pH and temperature, can be controlled during the culturing period. Those of ordinary skill in the art will be aware of, and will be able to select, suitable bioreactors for use in practicing the method described herein.

The method of the disclosure can be conducted using single-use bioreactors, also known as disposable bioreactors, which utilize disposable bags instead of traditional culture vessels. Shifting to single-use technology minimizes infrastructure requirements associated with traditional cell culture, such as steel/glass industrial scale vessels and associated machinery. Single-use bioreactors provide flexibility to the manufacturing process; and site assembly, reconfiguration, sterilization, and validation are faster, easier, and less costly than traditional cell culture plants. Single-use bioreactors typically utilize disposable, plastic sterile bags supported by a non-disposable support structure. The culture is agitated by stirrer within the bag or by rocking, and sensors measure and adjust various parameters of the culture, such as pH, temperature, oxygen, cell density, and the like. Single-use bioreactors are commercially available from, for example, Xcellerex, GE, Hyclone and Sartorius.

The bioreactor system maintains conditions within the bioreactor to support cell culture. Suitable culture conditions for mammalian cells are known in the art. See e.g., Animal cell culture: A Practical Approach, D. Rickwood, ed., Oxford University Press, New York (1992). By “running” a bioreactor system is meant maintaining conditions in the bioreactor system to support cell culture. A bioreactor “run” typically comprises the steps of inoculating a prepared bioreactor with a seed culture, and growing the culture for a suitable or predetermined time until the culture is terminated, usually by harvesting the contents of the bioreactor. For production bioreactor (N bioreactor) runs, a seed bioreactor or N−1 bioreactor is typically used to grow the cells used for the inoculation of the production bioreactor.

“Culturing” refers to maintaining cells in culture medium under conditions suitable for the survival and/or proliferation of the cells apart from a multicellular organism or tissue, and for producing the protein product. Cell cultures are typically operated in batch, fed batch, or perfusion modes. In batch mode, a fixed amount of culture medium and cells are added to the bioreactor at the start of the run. Over the course of the culture, the media volume in the reactor remains constant while the nutrient content of the media decreases. The cell concentration increases over the course of the run and may plateau and decline as nutrient content is depleted and waste products increase. Fed batch culture, like batch culture begins with an inoculation of cells and a fixed amount of culture media. Unlike batch culture, the volume of media in the bioreactor increases as concentrated nutrients are added over the course of the culture.

A “growth” cell culture medium or feed medium refers to a cell culture medium that is typically used in cell cultures during a period of exponential growth, a “growth phase”, and is sufficiently complete to support the cell culture during this phase. A growth cell culture medium may also contain selection agents that confer resistance or survival to selectable markers incorporated into the host cell line. Such selection agents include, but are not limited to, geneticin (G4118), neomycin, hygromycin B, puromycin, zeocin, methionine sulfoximine, methotrexate, glutamine-free cell culture medium, cell culture medium lacking glycine, hypoxanthine and thymidine, or thymidine alone. Growth cell culture media are known in the art.

A “production” cell culture medium or feed medium refers to a cell culture medium that is typically used in cell cultures during the transition when exponential growth is ending and during the subsequent transition and/or production phases when protein production takes over. Such cell culture medium is sufficiently complete to maintain a desired cell density, viability and/or product titer during this phase. Production cell culture media are known in the art.

Perfusion culture, like batch culture begins with a fixed inoculation of cells and culture media. Unlike batch and fed-batch culture, fresh feed medium is added or perfused into the bioreactor and an equivalent amount of spent media is withdrawn. In the case of the methods described herein, the perfusion is a continuous perfusion. A retention device, such a tangential flow filtration (TFF) system, an alternating tangential flow (ATF) system or recirculating tangential flow (RTF) system may be used to remove the spent media and unwanted by-products from the bioreactor.

RTF relies on use of a recirculation means, most commonly a peristaltic pump, to move cell culture uni-directionally and parallel to a membrane surface to allow removal of spent media while retaining cells in the bioreactor. ATF systems are similar to those of RTF systems with the exception of a pump which moves cell culture back and forth through the module (e.g., through hollow-fiber filter modules), instead of flowing in only one direction. See, e.g., U.S. Pat. No. 6,544,424; Furey (2002) Gen. Eng. News. 22 (7), 62-63. A benefit of ATF is a cleaning effect on the filter that is induced by the alternating flow. An exemplary method of the disclosure comprises running an (N) bioreactor using an ATF perfusion system.

Typically, hollow fiber filters are used in the RTF or ATF system (although this is not required). When the cell culture, including cell culture media, cells (whole and lysed), soluble expressed recombinant proteins, host cell proteins, waste products and the like, are introduced to the filter, depending on the pore size or molecular weight cutoff (MWCO), the hollow fiber material may retain certain cell culture components (in addition to the cells, themselves) on the lumen side (inside) and allow certain components to pass through the filter (permeate) based on the pore size or molecular weight cutoff of the hollow fiber material. The material that is retained (retentate) is returned to the bioreactor. Fresh perfusion cell culture media is added to the bioreactor and permeate is withdrawn from the filter at predetermined intervals or continuously to maintain a desired or constant bioreactor volume. The permeate can be discarded, stored in holding tanks, bags or totes or transferred directly to another unit operation, such as filtration, flocculation, centrifugation and/or other downstream purification methods or the like.

Hollow fibers, in various aspects, have inner diameters of about 0.5 mm to about 1 mm, and may be of any suitable length (e.g., about 30 cm to about 110 cm). Hollow fibers for microfiltration typically have a pore size ranging from 0.1 μm to 10 μm or a molecular weight cut off of 500 to 750 kDa or more and can be used to allow the protein to pass through into the permeate. Ultrafiltration hollow fibers typically have a pore size range of 0.01 μm to 0.1 μm or a molecular weight cut off of 300 kDa or less and can be used to retain the desired protein in the retentate and return it back to the bioreactor. This can be used, for example, to concentrate a recombinant protein product for harvest. Such filters are available commercially, such as Xampler UFP-750-E-4MA, Xampler UFP-30-E-4MA, (GE Healthcare, Pittsburgh, Pa.) and Midikros TC Modules T02-E030-10, T02-050-10, T02-E750-05, T02-M10U-06 (Spectrum Laboratories, Inc, Dominguez, Calif.), XCell ATF®, Repligen, Waltham, MA).

Cell culture fluid may be drawn out of the bioreactor and into a filter module by a pumping system, which passes the cell culture through or along a filter (e.g., through the lumen side of the hollow fiber). Examples of cell pumping systems include peristaltic pumps, double diaphragm pumps, low shear pumps (Levitronix™ pumps, Zurich, Switzerland), and alternating tangential flow systems (ATF™, Repligen, Waltham, MA). The permeate may be drawn from the filters by use of peristaltic pumps. In the examples, perfusion is accomplished by use of an alternating tangential flow system.

The methods of the present disclosure can be used as part of a larger production process whereby cells are cultured in three or more distinct phases. For example, cells may be cultured in one or more growth phases prior to the N−1 production phase, cultured in the N−1 production phase, then transferred to a (N) production phase under conditions that maximize protein production. Each phase can be conducted in its own bioreactor vessel or other vessel suitable for cell culture. Alternatively, more than one phase can be conducted in a common vessel. In one such example, a growth phase and a production phase are conducted in the same bioreactor vessel. In a commercial process for production of a protein by mammalian cells, there are commonly multiple, for example, at least about 2, 3, 4, 5, 6, 7, 8, 9, or 10 growth phases that occur in different culture vessels preceding a final production phase.

The duration of the N−1 stage can range from, e.g., 3 to 14 days, and can be designed so as to maintain cells in exponential growth prior to inoculation of a production (N) bioreactor.

Cell culture medium is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Cell culture media can be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

Cell culture media can be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162.

Defined cell culture media formulations are complex, containing amino acids, inorganic salts, carbohydrates, lipids, vitamins, buffers, and trace essential elements. Identifying the components that are necessary and beneficial to maintain a cell culture with desired characteristics is an on-going task. Defined basal media formulations which are supplemented or enriched to meet the needs of a particular host cell or to meet desired performance parameters is one approach to developing defined media.

### Inducing Production Phase

Bioreactor process parameters that can be used to switch to a non-steady state condition are permeate rate and temperature. Combinations of permeate rate, temperature and biomass can be used to favor protein production over cell growth and result in non-steady state CM operation. These parameters are empirically determined for each cell line to maximize productivity.

A cell culture temperature is typically 35° C. to about 38° C. Cell cultures typically contain at least one exponential growth phase and may contain a production phase. A growth phase can occur at a higher temperature than a production phase. For example, a growth phase can occur at a first temperature from about 35° C. to about 37° C., and to induce a non-steady state, a production phase can occur at a second temperature which is lower than the first temperature, for example, from about 29° C. to about 37° C., from about 30° C. to about 36° C., from about 32° C. to about 36° C. or from about 30° C. to about 34° C. In addition, chemical inducers of protein production, such as, for example, caffeine, sodium butyrate, and hexamethylene bisacetamide (HMBA), can be added at the same time as, before, and/or after a temperature shift. If inducers are added after a temperature shift, they can be added from one hour to five days after the temperature shift, optionally from one to two days after the temperature shift. A growth phase can also occur at a higher pH than a production phase.

There can be a lowering of permeate rate to about 1.0 to about 2.5 working volumes/day to induce a non-steady state.

Various media formulations can be used during the life of the culture, for example, to facilitate the transition from one stage (e.g., the growth stage or phase) to another (e.g., the production stage or phase) and/or to optimize conditions during cell culture (e.g., concentrated media provided during perfusion culture). A growth medium formulation can be used to promote cell growth and minimize protein expression. A production medium formulation can be used to promote production of the protein of interest and maintenance of the cells, with minimal cell growth. A feed medium, typically a media containing more concentrated components such as nutrients and amino acids, which are consumed during the course of the cell culture may be used to supplement and maintain an active culture, particularly a culture operated in fed batch. A “perfusion” medium refers to a feed medium that is specifically developed for use in cell cultures that are maintained by perfusion methods and is sufficiently complete to support the culture during the process. Perfusion media formulations are typically richer, more concentrated and continuously fed at higher total volumes over the culture period than basal culture medium and fed-batch feed media formulations to accommodate the higher cell densities and the method used to remove the spent medium. Perfusion medium can be used during both the growth and production phases of the culture. Such a concentrated feed medium and perfusion medium typically contain those components that have been depleted and/or are necessary to sustain the culture and can be present in the concentrated cell culture medium at, for example, about 2×, 3×, 4×, 5×, 6×, 7×, 8×, 9×, 10×, 12×, 14×, 16×, 20×, 30×, 50×, or more of their normal amount in basal media.

Culture pH is controlled at a preferred pH, typically about 6 to 7.4, preferably pH 6.85 to 7.2. In one embodiment the pH is 6.90 to 6.95. pH can be controlled by sparged CO₂ and 1M sodium carbonate. Dissolved oxygen is preferably 40 to 88 mmHg, more preferably 60-70 mmHg. Antifoam addition may be added at a frequency and volume suitable for the culture operation, supplementary bolus antifoam may be added as needed.

“Cell” or “cells” include any prokaryotic or eukaryotic cell. Cells include “host cells”, also referred to as “cell lines”, which are genetically engineered to express a protein of interest. Host cells are typically derived from a lineage arising from a primary culture that can be maintained in culture for an unlimited time. Genetically engineering the host cell involves transfecting, transforming or transducing the cells with a recombinant polynucleotide molecule, and/or otherwise altering (e.g., by homologous recombination and gene activation or fusion of a recombinant cell with a non-recombinant cell) to cause the host cell to express a desired protein. Methods and vectors for genetically engineering cells and/or cell lines to express proteins of interest are well known to those of skill in the art.

A host cell can be a eukaryotic cell, such as a mammalian cell. Any mammalian cell suitable for recombinant protein expression is appropriate for use in the context of the disclosure. Suitable mammalian cells include, but are not limited to, Chinese hamster ovary (CHO) cells, human embryonic kidney (HEK) cells, murine myeloma (NS0, Sp2/0) cells, baby hamster kidney (BHK) cells, human embryonic kidney (293) cells, fibrosarcoma (HT-1080) cells, human embryonic retinal (PER.C6) cells, hybrid kidney and B cells (HKB-11), CEVEC's amniocyte production (CAP) cells, human liver (HuH-7) cell, and any other cells that are used or suitable for use in clinical and/or commercial manufacturing. The most commonly used cell lines are from CHO cells. CHO cells are widely used to produce complex recombinant proteins. The dihydrofolate reductase (DHFR)-deficient mutant cell lines (Urlaub et al. (1980), Proc Natl Acad Sci USA 77:4216-4220), DXB11 and DG-44, are desirable CHO host cell lines because the efficient DHFR selectable and amplifiable gene expression system allows high level recombinant protein expression in these cells (Kaufman R. J. (1990), Meth Enzymol 185:537-566). The glutamine synthetase (GS)-knockout CHOK1SV cell lines, making use of glutamine synthetase (GS)-based methionine sulfoximine (MSX) selection are also widely used. Also included are CHOK1 cells (ATCC CCL61). Critical attributes and performance parameters of the cell line can be measured to better inform decisions regarding performance of each step during manufacture. These critical attributes and parameters can be monitored real-time, near real-time, and/or after the fact. Key critical parameters such as media components that are consumed (such as glucose), levels of metabolic by-products (such as lactate and ammonia) that may accumulate in the culture, as well as those related to cell maintenance and survival, such as dissolved oxygen content can be measured during the cell culture. Critical attributes such as specific productivity, viable cell density, pH, osmolality, appearance, viability, aggregation, cell count, packed cell volume, product quality, percent yield and titer may be monitored during appropriate stages in the manufacturing process. Process and product impurities may also be monitored throughout the manufacturing process.

Monitoring and measurements can be done using known techniques and commercially available equipment. Detection of product quality attributes can be achieved using mass spectrometry, liquid chromatography with UV and/or mass spectrometry detection, capillary electrophoresis, and the like. Post-translational modifications such as amino acid processing and glycosylation can be characterized using, for example, a polyhydroxyethyl aspartamide column operated in size-exclusion mode and coupled with ESI-MS (Brady et al., (2008) J Am Soc Mass Spectro, 19:502-509). Real-time monitoring of eluate from ion exchange chromatography can be performed by monitoring a normalized LS/UV ratio for each fraction using laser light scattering detector and an UV absorbance (see, e.g., U.S. Patent Publication No. US20130303732).

For example, viable cell density (VCD) and viability (%) may be determined using a Cedex HiRes (Roche, Basel, Switzerland). Glucose, lactate, and NH4+ concentrations may be determined using a Cedex BioHT (Roche, Basel, Switzerland). Titers may be determined by high performance liquid chromatography (HPLC) via affinity chromatography (Protein A, Waters, Milford, MA). The percentage of impurities may be determined using reduced capillary electrophoresis sodium dodecyl sulfate (rCE-SDS), non-reduced capillary electrophoresis sodium dodecyl sulfate (nrCE-SDS), ultra high-performance liquid chromatography (SE-UHPLC), acidic and basic charged variant species using cation exchange chromatography (CEX-HPLC).

The cell culture viscosity, including components of the media and cells themselves, can be between about 1 to about 6 centipoise (e.g., about 2 to about 6 centipoise). Cell culture viscosity can be characterized using any suitable viscometer, such as a “cone-and-plate” viscometer. The cell culture density, including components of the media and cells themselves, can be from about 1 g/L to about 1.5 g/L. Cell culture density can be characterized by, e.g., using an automated cell counter, such as the Roche Cedex HiRes, which uses a Trypan Blue Exclusion method.

### Harvesting

Methods provided herein can further comprise harvesting the protein product from the cell culture. In a harvesting step, the protein product is separated from cell debris and production cells in the culture medium. If desired, the bioreactor contents can be chilled for the harvest step. For example, the temperature of the bioreactor contents can be reduced to less than 12° C. (but greater than 0° C.). Harvest can be performed by any suitable method, including acid precipitation, accelerated sedimentation such as flocculation, separation using gravity, centrifugation, acoustic wave separation, filtration, including membrane filtration using ultrafilters, microfilters, tangential flow filters, including use of tangential flow filters in alternative tangential flow and recirculating tangential flow, depth filters, and alluvial filters. Depth filtration may be part of the harvest process to provide additional removal of impurities. One or more depth filters of the same or different materials, natural and/or synthetic may be used. Depth filters commonly used in biomanufacturing processes are typically composed of cellulose or polypropylene fibers, diatomaceous earth or perlite, or charged resin. The depth filter may optionally include a filter membrane layer of varying pore size, such as 0.22 μm.

### Downstream Purification

The harvested protein can be further purified away from any impurities, such as remaining cell culture media, cell extracts, host cell proteins, DNA, viruses, improperly expressed proteins, product-related impurities, and the like through one or more downstream purification processes. The downstream process operations can be performed in batch, semi-continuous and/or continuous mode. Two or more operations may have a direct connection, for example with surge tanks, holding tanks, bags, or other suitable containers adapted to accept a feed from at least one operation to another operation.

Capture chromatography is often used as an initial purification step followed by one or more intermediate and/or polish chromatography steps.

Affinity chromatography is commonly used as an initial capture step for harvested recombinant proteins as it performs well for purification of crude or clarified material. Affinity chromatography medium can comprise a substrate-binding capture mechanism, an aptamer-binding capture mechanism, or a cofactor-binding capture mechanism, for example. For proteins containing an Fc component, substrate-binding capture mechanism such as Protein A, Protein G, Protein A/G, and Protein L can be used. A multitude of Protein A affinity chromatography resins and materials are commercially available including, but not limited to, MabSelect™ from GE Healthcare, PROSEP® Ultra Plus from Millipore and Praesto® APc+ from Purolite. An exemplary resin is MabSelect™ SuRe resin (GE Healthcare Life Sciences), which exhibits enhanced clearance of low molecular weight species (LMWS).

One or more intermediate and/or polish chromatography steps remove any remaining contaminants and/or impurities. An intermediate and/or polish chromatography step makes use of chromatography media, such as resins, monoliths and/or membranes, containing agents that can be used in either a bind and elute mode (where the protein of interest is bound to the chromatography medium and eluted after the contaminants and impurities have flowed through or been washed off the chromatography medium), frontal or overloaded mode (where a solution containing the protein of interest is loaded onto a column until adsorption sites on are occupied and the species with the least affinity for the stationary phase (the protein of interest) starts to elute), a flow-through mode (where the protein of interest flows through the chromatographic material without binding and the contaminants and impurities are bound to the chromatography medium), or by any other suitable mode or a combination of modes. Examples of the most common chromatography modalities used for the intermediate and/or polish steps for biologic manufacturing include, but are not limited to, ion exchange chromatography (IEX), such as anion exchange chromatography (AEX) and cation exchange chromatography (CEX); hydrophobic interaction chromatography (HIC); mixed modal or multi-modal anion exchange chromatography (MMC); and hydroxyapatite chromatography (HA). Each of the polish chromatography unit operations may be run in the same or different configurations and/or different modes.

Each chromatography unit may be run as a single unconnected unit, multiple connected units, and/or combined units. Single chromatography columns may be run in a staggered cycling system, counter current loading (periodic counter current chromatography), or as a multicolumn counter current solvent gradient purification process (MCSGP), for example.

Chromatography mediums are well known and common in the art and are commercially available from many sources. Cation exchange mediums include, but are not limited to, those comprising a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. CEX resins include, but not limited to, Mustang S, Sartobind S, SO3 Monolith, S Ceramic HyperD, Poros XS, Poros HS50, Poros HS20, SPSFF, SP-Sepharose XL (SPXL), CM Sepharose Fast Flow, SP Sepharose Fast Flow XL™, SP-Sepharose High Performance™, Capto S, Capto SP ImpRes™, TOYOPEARL® HS, TOYOPEARL® XS, UNOsphere™, FractoPrep™, Fractogel Se HiCap, Fractogel SO₃, or Fractogel COO. Anion exchange mediums include, but are not limited to Source 15Q, Capto™ Q, Q-sepharose Fast Flow™, Fractogel EDM TMEA™, Fractogel EDM DEAE, TOYOPEARL Super Q®, Poros HQ™, and POROS XQ™. Mixed mode or multimode mediums include Capto™ Adhere. Hydrophobic interaction chromatography materials including, but not limited to, Fractogel™ EMD Propyl or Fractogel™ EMD Phenyl columns (Merck), Octyl Sepharose™ High Performance column (Pharmacia LKB Biotechnology), Phenyl Sepharose™ 6 Fast Flow column with low or high substitution (Pharmacia LKB Biotechnology), Phenyl Sepharose™ High Performance column (Pharmacia LKB Biotechnology), Macro-Prep™ Methyl or Macro-Prep™ t-Butyl Supports (Bio-Rad). WP HI-Propyl (C₃)™ column (J. T. Baker), and Toyopearl™ ether, phenyl or butyl columns (TosoHaas).

### Virus Inactivation and Virus Filtration

Viral mitigation measures are critical to ensure the safety of protein therapeutics. Viral contaminants can arise from a variety of sources including use of reagents of animal origin, adventitious viral contaminants in host cell lines, or system failures at GMP manufacturing sites. Viruses are classified as enveloped and non-enveloped viruses. Enveloped viruses have a capsid enclosed by a lipoprotein membrane or “envelope” made up of host cell proteins and phospholipids as well as viral glycoproteins which coat the virus as it buds from its host cell. This envelope allows the virus to identify, bind, enter, and infect target host cells. As such, enveloped viruses are susceptible to inactivation methods. Non-enveloped viruses are more difficult to inactivate without risk to the protein being manufactured and are removed by filtration methods. Viral mitigation strategies may be performed one or more times throughout the downstream purification.

A variety of methods are employed for virus inactivation and include heat inactivation/pasteurization, UV and gamma ray irradiation, use of high intensity broad spectrum white light, addition of chemical inactivating agents, and surfactants. Low pH and solvent/detergent treatments are the most common viral inactivation methods in manufacturing processes for protein therapeutics. Viral inactivation is typically performed following purification of the harvest fluid with affinity chromatography, in particular affinity chromatography that makes use of a substrate binding ligand from Staphylococcus aureus, such as Protein A chromatography, since elution from such a chromatography material is usually performed at low pH. The acidified eluant is held for an amount of time that has been determined to inactivate the virus concentration by the required number of logs. This is followed by neutralization of the inactivated material. Exemplary low pH viral inactivation methods are described in U.S. Application 63/168,608 and U.S. Application 63/159,217. An exemplary detergent inactivation is described in International Patent Publication No: WO 2020/190985.

Non-enveloped viruses are difficult to inactivate without risk to the recombinant product; however, such viruses can be removed by size-based filtration methods. Pre-filters can be used in combination with viral filters to help eliminate certain contaminants in the product pool or eluate stream before applying the pool or eluate to the viral filter to maintain continuity flow during the virus filtration operation. An exemplary process is described in International Patent Publication No: WO2020/159838. Viral filtration can be performed using micro- or nano-filters, such as those available from PLAVONA® (Asahi Kasei, Chicago, IL), VIROSART® (Sartorius, Goettingen, Germany), VIRESOLVE® Pro (MilliporeSigma, Burlington, MA), Pegasus™ Prime (Pall Biotech, Port Washington, NY), CUNO Zeta Plus VR, (3M, St. Paul, Mn). Viral filtration may occur at one or more steps in the downstream operations of a biomanufacturing process. Typically, viral filtration precedes the UFDF operation, but may also take place following UFDF.

### UF/DF

The method further optionally comprises concentrating the protein product using ultrafiltration and diafiltration (UFDF). The purified protein is subjected to an ultrafiltration and diafiltration operation comprising concentrating or diluting the purified protein by ultrafiltration; buffer exchanging the purified concentrated/diluted protein into a desired formulation by diafiltration; further diluting or concentrating the formulated purified protein by a second round of ultrafiltration until a target protein concentration is achieved. One or more stability-enhancing excipients can be added directly to the UFDF retentate feed tank containing the formulated purified protein resulting in formulated drug substance, or added to the UFDF eluate pool. Filters for use in a UFDF operation are well known and common in the art and are commercially available from many sources. There are many types of materials available, regenerated cellulose Pellicon (MilliporeSigma, Danvers, MA), stabilized cellulose, Sartocon® Slice, Sartocon® ECO Hydrosart® (Sartorius, Goettingen, Germany), polyethersulfone (PES) membrane, Omega (Pall Corporation, Port Washington, NY). Multiple filters can be used to the capacity that holders, skids, or the physical set up of the UFDF system will allow or are needed to achieve the desired objectives of a production process.

### Biopharmaceuticals

Methods provided herein can be used to produce a protein product such as a recombinant protein. The recombinant protein can be a eukaryotic protein, such as a mammalian protein.

The mammalian protein can be an antigen-binding protein, such as an antibody, an antibody fragment, an antibody derivative, an antibody analog, an antibody construct, a fusion protein, a mutein, a multispecific protein, a bispecific protein, a bispecific T-cell engager, or a peptibody. The antibody can be a whole antibody, a single-chain variable fragment, a Fv, a Fab, a Fab′, a F(ab′) 2, a bispecific antibody, a diabody, a triabody, a tetrabody, a Fd, a dAb, a minibody, or a maxibody.

“Multispecific proteins” and “multispecific antibodies” refer to proteins that are recombinantly engineered to simultaneously bind at least two different antigens or at least two different epitopes on the same antigen. For example, multispecific proteins can be engineered to target immune effectors and cytotoxic agents to tumors or infectious agents. These multispecific proteins have been found useful for a variety of applications such as in cancer immunotherapy by redirecting immune effector cells to tumor cells, modifying cell signaling by blocking signaling pathways, targeting tumor angiogenesis, blocking cytokines, and as pre-targeted delivery vehicles for drugs, such as delivery of chemotherapeutic agents, radiolabels (to improve detection sensitivity) and nanoparticles (directed to specific cells/tissues, such as cancer cells).

The most common and diverse of the multispecific proteins are those that bind two antigens, referred to interchangeably herein as “bispecific protein” and “bispecific antibody”. Multispecific proteins also include trispecific antibodies, tetravalent bispecific antibodies, multispecific proteins without antibody components such as dia-, tria- or tetrabodies, minibodies, and single chain proteins capable of binding multiple targets. Coloma, M. J., et. al., Nature Biotech. 15 (1997) 159-163.

Bispecific proteins can be grouped in two broad categories: immunoglobulin G (IgG)-like molecules and non-IgG-like molecules. IgG-like molecules retain Fc-mediated effector functions, such as antibody-dependent cell mediated cytotoxicity (ADCC), complement-dependent cytotoxicity (CDC), and antibody-dependent cellular phagocytosis (ADCP), the Fc region helps improve solubility and stability and facilitate some purification operations. Non-IgG-like molecules are smaller, enhancing tissue penetration. (Sedykh et al., Drug Design, Development and Therapy 18 (12), 195-208, 2018; Fan et al., J Hematol & Oncology 8:130-143, 2015; Spiess et al., Mol Immunol 67, 95-106, 2015); Williams et al., Chapter 41 Process Design for Bispecific Antibodies in Biopharmaceutical Processing Development, Design and Implementation of Manufacturing Processes, Jagschies et al., eds., 2018, pages 837-855. Bispecific proteins are sometimes used as a framework for additional components having binding specificities to different antigens or numbers of epitopes, increasing the binding specificity of the molecule.

Bispecific proteins come in a variety of formats, including but are not limited to, quadromas, knobs-in-holes, cross-Mabs, dual variable domains IgG (DVD-IgG), IgG-single chain Fv (scFv), scFv-CH3 KIH, dual action Fab (DAF), half-molecule exchange, KA-bodies, tandem scFv, scFv-Fc, diabodies, single chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), XmAb®; Fab-arm exchange, DutaMab, DT-IgG, charged pair, Fcab, orthogonal Fab, IgG(H)-scFv, scFV-(H) IgG, IgG(L)-scFV, IgG(L1H1)-Fv, IgG(H)-V, V(H)-IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, single chain bispecific antibody constructs, single chain bispecific T cell engagers (scBiTE), bi-specific T cell engagers (BiTE®), and half-life extended bispecific T cell engagers (HLE BITE) (Fan supra; Spiess supra; Sedykh supra; Seimetz et al., Cancer Treat Rev 36 (6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p559-594, John Wiley & Sons, 2017; Moore et al., MAbs 3:6, 546-557, 2011

Methods provided herein can be used to produce a colony-stimulating factor, an erythropoiesis stimulating agent, a HER receptor, a cell adhesion molecule, a growth factor, an osteoinductive factor, insulin, a coagulation protein, a colony stimulating factor, a blood group antigen; a growth hormone, a growth hormone receptor, a T-cell receptor; a neurotrophic factor, a neurotrophin, a relaxin, an interferon, an interleukin, a viral antigen, a lipoprotein, an integrin, a rheumatoid factor, an immunotoxin, a surface-membrane protein, a transport protein, a homing receptor, an addressin, a regulatory protein, or an immunoadhesin. The growth factor can be a nerve growth factor, a fibroblast growth factor, a transforming growth factor, or an insulin-like growth factor. The colony stimulating factors can be a granulocyte colony-stimulating factor (G-CSF). Such G-CSF molecules include, but are not limited to, Neupogen® (filgrastim) and Neulasta® (pegfilgrastim). Also included are erythropoiesis stimulating agents (ESA), such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin®(epoetin theta), epoetin alfa, epoetin beta, epoetin zeta, epoetin theta, and epoetin delta, epoetin omega, epoetin iota, tissue plasminogen activator, GLP-1 receptor agonists, as well as the molecules or variants or analogs thereof and biosimilars of any of the foregoing.

Methods provided herein can be used to produce proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

Methods provided herein can be used to produce CD proteins, including but not limited to, CD3, CD4, CD5, CD7, CD8, CD19, CD20, CD22, CD25, CD30, CD33, CD34, CD38, CD40, CD70, CD123, CD133, CD138, CD171, and CD174; HER receptor family proteins, including but not limited to, HER2, HER3, HER4, and the EGF receptor, EGFRvIII; cell adhesion molecules, including but not limited to, LFA-1, Mol, p150,95, VLA-4, ICAM-1, VCAM, and alpha v/beta 3 integrin; growth factors, including but not limited to, vascular endothelial growth factor (“VEGF”), VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor (such as NGF-beta), platelet-derived growth factor (PDGF), fibroblast growth factors [including but not limited to, aFGF and bFGF], epidermal growth factor (EGF), Cripto, transforming growth factors (TGF) (including, among others, TGF-α and TGF-β, including TGF-β1, TGF-β2, TGF-β3, TGF-β4, or TGF-β5), insulin-like growth factors-I and -II (IGF-I and IGF-II), des (1-3)-IGF-I (brain IGF-I), and osteoinductive factors; insulins and insulin-related proteins, including but not limited to, insulin, insulin A-chain, insulin B-chain, proinsulin, and insulin-like growth factor binding proteins; coagulation and coagulation-related proteins, such as, among others, factor VIII, tissue factor, von Willebrand factor, protein C, alpha-1-antitrypsin, plasminogen activators [such as urokinase and tissue plasminogen activator (“t-PA”)], bombazine, thrombin, thrombopoietin, and thrombopoietin receptor; colony stimulating factors (CSFs), including but not limited to, M-CSF, GM-CSF, and G-CSF; other blood and serum proteins, including but not limited to; albumin, IgE, and blood group antigens; receptors and receptor-associated proteins, including but not limited to, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin; interferons, including for example, interferon-alpha, -beta, and -gamma; interleukins (ILs) including but not limited to, IL-1 to IL-10, IL-12, IL-15, IL-17, IL-23, IL-12/IL-23, IL-2Ra, IL1-R1, IL-6 receptor, IL-4 receptor, IL-13, IL-13RA2, orIL-17 receptor, IL-1RAP, IL1-α, and IL-1β); viral antigens, including but not limited to, an AIDS envelope viral antigen; lipoproteins; calcitonin; glucagon; atrial natriuretic factor; lung surfactant; tumor necrosis factor-alpha and -beta; enkephalinase; BCMA; IgKappa; ROR-1; ERBB2; mesothelin; RANTES (regulated on activation normally T-cell expressed and secreted); mouse gonadotropin-associated peptide; Dnase; FR-alpha; inhibin; activing; integrin; protein A or D; rheumatoid factors; immunotoxins; bone morphogenetic protein (BMP); superoxide dismutase; surface membrane proteins; decay accelerating factor (DAF); AIDS envelope proteins; transport proteins; homing receptors; MIC (MIC-a, MIC-B); ULBP 1-6; EPCAM; PSA; addressins; regulatory proteins; immunoadhesins; antigen-binding proteins; somatropin; CTGF; CTLA4; eotaxin-1; MUC1; CEA; c-MET; Claudin-18; GPC-3; EPHA2; FPA; LMP1; MG7; NY-ESO-1; PSCA; ganglioside GD2; ganglioside GM2; BAFF; OPGL (RANKL); myostatin; Dickkopf-1 (DKK-1); Ang2; NGF; IGF-1 receptor; hepatocyte growth factor (HGF); TRAIL-R2; c-Kit; B7RP-1; PSMA; P-cadherin; NKG2D-1; programmed cell death protein 1 and ligand (PD1 and PDL1); mannose receptor/hCGB; hepatitis-C virus; mesothelin dsFv; PE38 conjugate; Legionella pneumophila (IIy); gpA33; B7H3; IFN gamma; interferon gamma induced protein 10 (IP10); IFNAR; TALL-1; thymic stromal lymphopoietin (TSLP); proprotein convertase subtilisin/Kexin Type 9 (PCSK9); stem cell factors; Flt-3; calcitonin gene-related peptide (CGRP); OX40L; a4ß7; platelet specific (platelet glycoprotein lib/IIIb (PAC-1); transforming growth factor beta (TFGβ); Zona pellucida sperm-binding protein 3 (ZP-3); TWEAK; platelet derived growth factor receptor alpha (PDGFRa); sclerostin; and biologically active fragments or variants of any of the foregoing.

Methods provided herein can be used to produce abciximab, adalimumab, adecatumumab, aflibercept, alemtuzumab, alirocumab, anakinra, atacicept, basiliximab, belimumab, bevacizumab, biosozumab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, Ixdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, as well as variants or analogs thereof, and biosimiliars of any of the foregoing.

Methods provided herein can be used to produce blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813 (RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; BII034020, AFM13, AFM11, SAR156597, FBTA05, PF06671008, GSK2434735, MEDI3902, MEDI0700, MEDI7352, as well as variants or analogs thereof, and biosimilars of any of the foregoing.

### Other Terms

While various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together.

When using about in connection with a numerical value, the disclosure contemplates a range around the numerical value. For instance, about can mean±1%, ±2%, ±5%, ±10%, etc. About can also mean±0.1 units, ±0.2 units, ±0.3 units, ±0.4 units, ±0.5 units, ±1 unit, when referring to a unit such as temperature or pH.

When describing a range of values, the disclosure contemplates individual values found within the range. For example, “cell aggregate size of between about 20 μm and about 200 μm,” could be, but is not limited to, 40 μm, 60 μm, 100 μm, etc., and any value in between such values. In any of the ranges described herein, the endpoints of the range are included in the range. However, the disclosure also contemplates the same ranges in which the lower and/or the higher endpoint is excluded. When a value is stated to be “about” a value, the exact value is also contemplated whether individually or as a endpoint of a range.

As used herein, “may be,” “may,” “can be,” or “can” indicates something envisaged by the inventors that is functional and available as part of the subject matter provided.

Additional features and variations of the invention will be apparent to those skilled in the art from the entirety of this application, including the figures and detailed description. The entire document is intended to be related as a unified disclosure, and it should be understood that all combinations of features described herein (even if described in separate sections) are contemplated, even if the combination of features is not found together in the same sentence, or paragraph, or section of this document.

### EXAMPLES

While the following examples describe specific embodiments, variations and modifications will occur to those skilled in the art. Accordingly, only such limitations as appear in the claims should be placed on the invention.

### Example 1

### Experiment 1

The first experiment illustrates non-steady state cell culture in the first condition. Within the first experiment, there were process conditions to induce non-steady state cell culture.

A 100 L single use bioreactor (Xcellerex, Marlborough, MA) was inoculated with CHO cells expressing an antigen-binding protein at a density of 45×10⁵ cells/ml in 60 L of serum-free, chemically defined basal medium. Following inoculation, the target volume in the bioreactor was adjusted to 100 L of basal medium, at a setpoint pH of 6.90 and an initial temperature of 36.0° C. and maintained for 24 hours.

The bioreactor was equipped with two ATF 6 (0.2 μm) alternating tangential flow devices (Repligen, Waltham, MA). On Day 1, the culture was continuously perfused with a serum-free, chemically defined perfusion medium and continued operation at pH 6.90 and a temperature of 36.0° C. The permeate flow rate was adjusted daily to reach a maximal working volume (WV) per day of 2.0 on Day 6, see Table 1. The cells remained in the retenate and were returned to the bioreactor to build and/or maintain cell density. Any recombinant product passed in the permeate flow and was discharged to waste during the growth phase (Days 0-6). The biomass-specific permeate rate was 0.0211 cm/pF·day at the end of the growth phase.

Cell count, viability, cell diameter, pH, pCO₂, pO₂, permittivity, glucose, lactate, ammonia, osmolality, packed cell volume, bioreactor titer, permeate titer, and harvest titer were measured daily. Bulk bioreactor supernatant and harvest samples were taken for PQ. Antifoam was added directly to the bioreactor to control foam.

TABLE 1
Permeate flowrate schedule

| Day | WV per Day |
|---|---|
| 1 | 0.5 |
| 2 | 0.8 |
| 3 | 1.2 |
| 4 | 1.5 |
| 5 | 1.8 |
| 6-28 | 2.0-2.2 |

TABLE 2
Process parameters for Experiment 1

| | | | | | Biomass-specific |
|---|---|---|---|---|---|
| | | | Permittivity | Permeate | permeate rate at |
| | Time | Temperature | Maximum | Rate | Permittivity Target |
| Phase | (days) | (° C.) | (pF/cm) | (wv/day) | (pF/cm · day) |
| Growth | 0-6 | 36 | 95 | 0-2.0 | 0.0211 |
| Biomass | 6-7 | 34 | 105 | 2.0 | 0.0190 |
| Intensification | | | | | |
| Condition 1 | 8-20 | 34 | 95 | 2.0-2.2 | 0.0211 |
| Condition 2 | 21-28 | 36 | 65 | 2.2 | 0.0338 |

The culture was maintained until a target permittivity of 95 pF/cm was achieved (Day 6) at which time a temperature shift from 36.0° C. to 34.0° C. was performed to control cell growth and increase protein production (Table 3). A capacitance probe was used to monitor cell biomass and trigger the temperature shift at the target permittivity value. The capacitance probe was also used to increase and maintain biomass during the growth and biomass intensification phase by controlling a cell bleed to remove cells from the bioreactor. The minimal packed cell volume was ≥25%.

Biomass Intensification: Starting on Day 6 and following the temperature shift marking the start of the production phase, the biomass increased to a maximum of 105 pF/cm. This high cell density at the start of the production phase maintains a high protein production rate in the run. The biomass-specific permeate rate was 0.0190 pF/cm at this maximum biomass.

Non-steady state CM: The process parameters in this run, namely the combination of temperature, biomass and permeate rate (or alternatively biomass-specific permeate rate) resulted in non-steady state CM, in which the permittivity target and cell bleeds were no longer maintained.

Experiment 1 Condition 1: This condition was carried out in the production phase, so recombinant product in the permeate flow was continuously harvested into sterile harvest bags. From Day 8 to Day 20, the temperature setpoint was maintained at 34.0° C. and the permeate rate was maintained at 2.0-2.2 wv/day. The cell culture was carried out in non-steady state operation, so the VCD and viability declined, the cell diameter and PCV increased. Initially, the permittivity was maintained at a target of 95 pF/cm with a cell bleed. After about a week, the permittivity decreased below this maximum of 95 pF/cm as expected since biomass decreases in non-steady state operation. The benefit of decreasing VCD/viability/PCV/permittivity/bleed/cell growth rate was stable and high protein production rate.

Experiment 1 Condition 2: On Day 21, the culture temperature was increased to 36.0° C., and a permittivity target of 65 pF/cm was established and maintained by cell bleed. The cell culture was also a non-steady state operation but reduced the rate of biomass decrease in culture by increasing the temperature. The permeate rate was maintained at 2.2 wv/day. The biomass-specific permeate rate was 0.0338 pF/cm·day. Recombinant product in the permeate flow was continuously harvested into sterile harvest bags. The culture was continued under these conditions until Day 28 at which time the culture was terminated. The protein production rate remained just as high as Condition 1 initially, but the production rate decreased in the last several days. This illustrates that easing off from the process parameters in Condition 1 results in lower productivity.

### Experiment 1 Results

The first experiment demonstrated biomass intensification and non-steady state operation, which resulted in a high protein production rate. Thus, the effect of biomass intensification and Condition 1 appeared to determine the rate of production for most of the run. The production rate fell in the last few days of culture as a result of Condition 2, which had a higher temperature and reduced the rate of biomass decrease.

### Experiment 2

The second experiment begins with steady state conditions initially followed by non-steady conditions with declining viabilities.

In a second experiment, the process design space was explored to demarcate the steady versus non-steady CM operation and the degrees of magnitude of each type of CM operation. Several conditions of successively decreasing temperature and increasing permittivity were tested during the production phase. The purpose was to determine which combination of temperature and biomass-specific permeate rate (permeate rate relative to the biomass in culture) could sustain cell growth and result in the maintenance of a high viability (steady-state culture) as opposed to conditions at which the viability started to decrease (non-steady state culture). No biomass intensification step was tested in this experiment.

CHO cells were inoculated into a 100 L bioreactor at a density of 45×10⁵ cells/ml in 60 L of serum-free, chemically defined basal medium. Following inoculation, the target volume in the bioreactor was adjusted to 100 L of the basal medium, at a setpoint pH of 6.90 at an initial temperature of 36.0° C. and maintained for 24 hours. The permeate flow rate was adjusted daily to reach a maximal working volume (WV) per day of 2.0 on Day 6, see Table 3.

TABLE 3
Process parameters for Experiment 2

| | | | | | Biomass-specific |
|---|---|---|---|---|---|
| | | | Permittivity | Permeate | permeate rate at |
| | Time | Temperature | Target | Rate | Permittivity |
| Phase | (days) | (° C.) | (pF/cm) | (wv/day) | Target(pF/cm · day) |
| Growth | 0-6 | 36 | 75 | 0-2.2 | 0.0293 |
| Condition 1 | 7-8 | 36 | 75 | 2.2 | 0.0293 |
| Condition 2 | 9-12 | 35.5 | 75 | 2.2 | 0.0293 |
| Condition 3 | 13-17 | 35 | 85 | 2.2 | 0.0259 |
| Condition 4 | 18-25 | 34.5 | 95 | 2.2 | 0.0232 |
| Condition 5 | 26-35 | 34.5 | 115 | 2.2 | 0.0191 |

The culture was maintained until a permittivity target of 75 pF/cm was achieved (Day 6), Table 3. The minimal packed cell volume was ≥25%. The biomass-specific permeate rate was 0.0293 pF/cm·day.

Experiment 2 Condition 1: The culture was maintained at 36.0° C., with a target permittivity of 75 pF/cm and a permeate rate of 2.2 wv/day until day 8. The biomass-specific permeate rate remained at 0.0293 pF/cm·day upon reaching the permittivity target.

Experiment 2 Condition 2: On Day 8, a temperature shift from 36.0° C. to 35.5° C. was performed to control cell growth and promote protein production. The permittivity target (75 pF/cm) and permeate rate (2.2 wv/day) were maintained until Day 12. The biomass-specific permeate rate remained at 0.0293 pF/cm·day upon reaching the permittivity target.

Experiment 2 Condition 3: On Day 13, the temperature was reduced to 35.0° C. and the target permittivity was increased to 85 pF/cm and maintained until Day 17. The permeate rate was maintained at 2.2 wv/day. The biomass-specific permeate rate increased to 0.0259 pF/cm·day upon reaching the permittivity target.

Experiment 2 Condition 4: On Day 18, the temperature was reduced to 34.5° C. and the target permittivity was increased to 95 pF/cm. The permeate rate was maintained at 2.2 wv/day. The biomass-specific permeate rate increased to 0.0232 pF/cm·day upon reaching the permittivity target.

Experiment 2 Condition 5: On Day 26, the temperature was maintained at 34.5° C. and the target permittivity was increased to 115 pF/cm. The permeate rate was maintained at 2.2 wv/day. The biomass-specific permeate rate increased to 0.0191 pF/cm·day upon reaching the permittivity target.

In Experiment 2 Conditions 1-3, steady state was maintained in the cell culture parameters and the production rate was low. As the temperature and biomass was increased (decrease in biomass-specific permeate rate) in Experiment 2 Conditions 4-5, the culture began to exhibit characteristics of non-steady state CM (i.e., declining viability). The culture in Conditions 4-5 still had some cell growth (as evidenced by a cell bleed) so the non-steady state characteristics were not as extreme as those tested in Experiment 1. Experiment 2 illustrates the transition between the steady state and non-steady state CM design space. The cell culture performance indicators that shows the first sign of non-steady characteristics in Conditions 4-5 is the declining viability and decreasing rate of cell bleed. The production rate also increases in Conditions 4-5 versus the steady state Conditions 1-3. Due to the steady state nature of many of the conditions tested (high cell growth rates), the production rate in Experiment 2 was about half that of Experiment 1.

### Experiment 3

The third experiment illustrates non-steady state conditions without the biomass intensification phase.

Experiment 3 repeated Experiment 2 growth phase and condition 1 without the biomass intensification phase. The purpose was to determine the effect of the biomass intensification phase on cell culture performance parameters.

CHO cells were grown as described in Experiment 1, except that no biomass intensification was performed. The 6-day growth phase at 36.0° C. and the 12-day production phase at 34.0° C. (Condition 1) were carried out at the same permittivity (95 pF/cm) and permeate rate (2.0). The biomass-specific permeate rate for both the growth phase and Condition 1 was 0.0210 pF/cm.

Although Experiment 3 was a non-steady state CM operation, the rate of viability decline was not as steep and the cell growth rate (as shown by bleed rate) exceeded that of Experiment 1. There was lower productivity in this experiment as compared to Experiment 1. This phenomenon can be attributed due to a lack of biomass intensification phase.

TABLE 4
Process parameters for Experiment 3

| | | | | | Biomass-specific |
|---|---|---|---|---|---|
| | | | Permittivity | Permeate | permeate rate at |
| | Time | Temperature | Maximum | Rate | Permittivity Target |
| Phase | (days) | (° C.) | (pF/cm) | (wv/day) | (pF/cm · day) |
| Growth | 0-6 | 36 | 95 | 0-2.0 | 0.0210 |
| Condition 1 | 7-18 | 34 | 95 | 2.0 | 0.0210 |

### Experiment 4

The fourth experiment repeats the first experiment and illustrates the repeatability of the non-steady state cell culture.

Experiment 4 was carried out as a non-steady state CM operation with a different permittivity target and permeate rate. The biomass-specific permeate rate was lower than Experiment 1, the permittivity target was higher and the permeate rate was lower. This experiment showed another example of high productivity in non-steady state CM culture, similar to Experiment 1.

CHO cells were grown as described in Experiment 1, with a higher permittivity (115 pF/cm) during the growth phase at a lower permeate rate (1.8 wv/day), see Table 5. The biomass-specific permeate rate was 0.0157 pF/cm.

TABLE 5
Process parameters for Experiment 4

| | | | | | Biomass-specific |
|---|---|---|---|---|---|
| | | | Permittivity | Permeate | permeate rate at |
| | Time | Temperature | Maximum | Rate | Permittivity Target |
| Phase | (days) | (° C.) | (pF/cm) | (wv/day) | (pF/cm · day) |
| Growth | 0-6 | 36 | 115 | 0-1.8 | 0.0157 |
| Biomass | 7-8 | 34 | 115 | 1.8 | 0.0157 |
| Intensification | | | | | |
| Condition 1 | 9-27 | 34 | 105 | 1.8 | 0.0171 |

On Day 7, the temperature was lowered to 34.0° C., the permittivity and permeate rate stayed the same for a biomass intensification. The biomass-specific permeate rate was 0.0157 pF/cm.

One Day 9, the permittivity was lowered to 105 pF/cm, the biomass-specific permeate rate was 0.0171 pF/cm.

### Example 1 Results

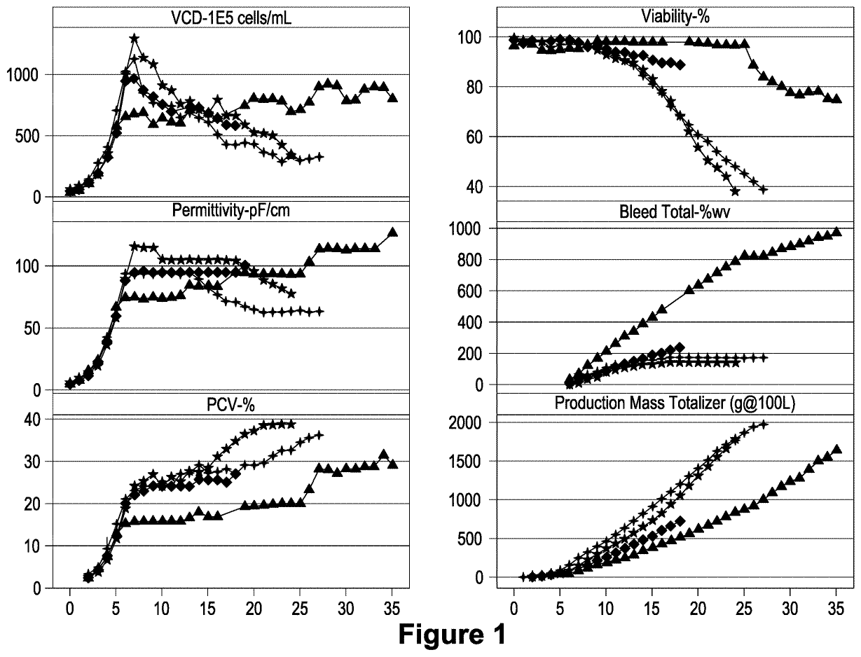

Experiment 1 demonstrated high protein production throughout the low temperature production phase. The production rate was similar throughout the process despite setpoint changes, See FIG. 1A-F. Thus, the biomass intensification phase and Condition 1 appeared to determine the high rate of protein production (shown in Production Mass Totalizer). In contrast, the effect of Condition 2 was only seen in the last few days, since it takes several days for the effects of a condition to manifest in the cell culture. Thus, the biomass intensification phase optimally needs to take place right after the growth phase, since this step in large part determines the protein production rate for the majority of the culture period.

The two examples of non-steady state CM with biomass intensification phase (Experiments 1 and 4) were tested for product quality in both lots of each run and compared to the single lot of a non-continuous perfusion process of the same cell line/molecule. Samples were taken from each run to determine the product quality attributes of the recombinant protein produced. These PQAs were compared to a non-continuous perfusion culture producing the antigen-binding protein. The PQAs were similar between lots of a non-steady state CM run. There did not appear to be an adverse impact to PQAs between different CM runs and between CM and a non-continuous perfusion process.

TABLE 6
Product quality attributes of two samples from
Experiment 1 compared to a non-continuous perfusion
process operated at steady state (Control).

| Attribute | Perfusion Run | CM Experiment 1 | | CM Experiment 4 | |
|---|---|---|---|---|---|
| PQA (%) | (Control) | Lot 1 | Lot 2 | Lot 1 | Lot 2 |
| SEC HMW | 19.9 | 22.6 | 23.9 | 18.04 | 15.42 |
| CEX Basic | 29.6 | 28.2 | 31.0 | 30.06 | 29.53 |
| CEX Acidic | 7.0 | 9.8 | 9.2 | 4.75 | 4.20 |
| rCE LMW | N/A | 2.9 | 3.5 | 1.85 | 2.39 |

There were two production lots from each CM bioreactor experimental run. The two lots within each experimental run were compared with respect to product quality. The product quality attributes were similar between the two CM lots from Experiment 1 and between the two CM lots of Experiment 2. The product quality was also fairly similar between the two different CM runs for most attributes. There was a non-continuous perfusion process carried out to produce the same protein from the same cell line. This process was also operated at a non-steady state but had different culture conditions (e.g., temperature, perfusion rate). The product was retained in the reactor and then perfused out in the last three days. The product quality attributes from the perfusion run were similar to the product quality attributes from the two CM runs despite the differences in process conditions and format.

Experiment 3 had a similar growth phase and first condition to Experiment 1, without the biomass intensification. The conditions of Experiment 3 resulted in lower production compared to Experiment 1, See FIG. 1A-F. This can be attributed to the lack of a biomass intensification phase in Experiment 3. Thus, the presence of the biomass intensification phase is important to ensure high productivity.

Experiment 4 had both the growth phase and biomass intensification phase followed by a production phase at the same temperature as Experiments 1 and 3. The permittivity and permeate setpoint were adjusted for a lower biomass-specific permeate rate. The productivity of Experiments 1 and 4 were similar due to similar phases of growth, biomass intensification and production, See FIG. 1A-F. This demonstrates that all three phases are important to establish high productivity in non-steady state CM culture.

The purpose of Experiment 2 was to determine which temperature and biomass-specific permeate rate setpoint could sustain cell growth and result in the maintenance of high viability (steady-state culture) and at which temperatures the viability started to decrease (non-steady state culture). Experiment 2 demonstrated that there is a process design space in which the conditions favor cell growth and a different design space which favors protein production. In the first three conditions, steady state conditions were observed and in the last two conditions, there were non-steady state conditions, as shown by the viability decrease See FIG. 1A-F.

### Overall Results of Example 1

There are several examples of conditions with non-steady state CM described above. There are different levels of biomass decrease with corresponding differences in production rates, which were the result of testing different process parameter combinations. The importance of the biomass intensification phase was demonstrated in Experiment 3 by removing this phase from culture and observing the impact on production rates. The transition between steady state CM and non-steady state CM was illustrated by gradually increasing biomass and decreasing temperature.

Experiments 1 and 4 demonstrate the non-steady state CM with the biomass intensification phase.

Viable cell density—The VCD increased in the growth phase (Days 0-6) and the biomass intensification phase (Days 6-8), peaked at Day 8 then steadily decreased in the remainder of the production phase (due to the non-steady state operation).

Viability—The viability also started to decrease in a similar fashion as VCD.

Permittivity—The permittivity declined in non-steady state but was often late to manifest non-steady characteristics as opposed to VCD and viability, which dropped immediately.

Bleed Total—The slope of the bleed total is a measure of cell growth. If the slope is horizontal, it indicates no cell growth. Sharper declines in viability correspond with lower bleed rates and low cell growth rates.

Packed cell volume—The PCV increases even as the VCD declines in non-steady state culture. This demonstrates that the cell diameter (and thus cell volume) increases in non-steady state culture.

Production Mass Totalizer—The slope of the production mass totalizer illustrates the protein production rate. This slope is steeper in non-steady state CM than steady state CM cultures.

### Example 1 Conclusion

Experiments 1~4 helped to define the non-steady state production phase. The process parameters that influence non-steady state behavior and their effects on culture were studied. Furthermore, the role of the biomass intensification phase in increasing productivity was elucidated. In Experiment 1, there was a biomass intensification phase, a first condition that resulted in non-steady state operation and a second condition that attempted to switch the process back to steady state operation. In Experiment 2, the culture was transitioned stepwise from steady state towards non-steady state by manipulating the biomass level and temperature. In Experiment 3, the process parameters that elicited non-steady state were repeated (i.e., Experiment 1, Condition 1 without the biomass intensification phase). This resulted in lower productivity, which is attributed to the absence of the biomass intensification phase. In Experiment 4, the non-steady state process (i.e., Experiment 1, Condition 1 with the biomass intensification phase) was repeated with similar process parameters as Experiment 1. This resulted in similar productivity as Experiment 1 and confirmed the non-steady state operation.

Operating at non-steady state (declining VCD, viability) cell culture resulted in much higher productivity than operating at steady state. This reduced the culture duration, and can improve plant utilization and thus the cost of the process. The harvest yield increased in culture due to a reduction in bleed volume. The harvested mass was higher due to the higher productivity and high permeate rates. Thus, non-steady state operation resulted in an economically favorable process. Product quality was similar across both lots of non-steady state CM, which demonstrates that the process was in a state of control.

### Example 2

Example 2 illustrates non-steady state cell culture on a different cell line and antigen-binding protein. This shows the repeatability of these methods. The conditions in this example had non-steady state cell culture.

A 100 L single use bioreactor (Xcellerex, Marlborough, MA) was inoculated with CHO cells expressing a second bispecific T cell engager at a density of 45×10⁵ cells/ml in 60 L of serum-free, chemically defined basal medium. Following inoculation, the target volume in the bioreactor was adjusted to 100 L of basal medium, at a setpoint pH of 6.95 and an initial temperature of 36.0° C. and maintained for 24 hours, as described in Example 1.

The bioreactor was equipped with two ATF 6 (0.2u) alternating tangential flow devices (Refine, Pine Brook, NJ). On Day 1, the culture was continuously perfused with a serum-free, chemically defined perfusion medium, pH 6.90, temperature 36.0° C. The permeate flow rate was adjusted daily to reach a maximal working volume (WV) per day of 1.8 by Day 5, see Table 7. Process parameters for the experiment are shown in Table 8. The cells remained in the retenate and returned to the bioreactor to build and/or maintain biomass. Any recombinant product passed in the permeate flow and was discharged to waste during the growth phase (Days 0-6). The biomass-specific permittivity was 0.0157 pF/cm·day.

Cell count, viability, cell diameter, pH, pCO₂, pO₂, permittivity, glucose, lactate, ammonia, osmolality, packed cell volume, bioreactor titer, permeate titer, and harvest titer were measured daily. Bulk bioreactor supernatant and harvest samples were taken for PQ.

TABLE 7
Permeate flowrate schedule.

| Day | WV per Day |
|---|---|
| 1 | 0.4 |
| 2 | 0.7 |
| 3 | 1.2 |
| 4 | 1.5 |
| 5-28 | 1.8 |

TABLE 8
Process parameter setpoints

| | | | | Permeate | Biomass-specific | |
|---|---|---|---|---|---|---|
| | Time | Temperature | Permittivity | Rate | permeate rate | Manual Bleed |
| Phase | (days) | (° C.) | (pF/cm) | (wv/day) | (pF/cm · day) | (%) |
| Growth | 0-6 | 36 | 115 | 0-1.8 | 0.0157 | N/A |
| Biomass | 7-8 | 34 | 115 | 1.8 | 0.0157 | N/A |
| Intensification | | | | | | |
| Condition 1 | 9-14 | 34 | 105 | 1.8 | 0.0171 | N/A |
| Condition 2 | 15-20 | 35 | 105 | 1.8 | 0.0171 | 5 |
| Condition 3 | 21-28 | 35 | 105 | 1.8 | 0.0171 | N/A |

The objective of this experiment was to demonstrate non-steady state operation for a different molecule and test the effect of the process conditions for a different molecule. The growth phase, biomass intensification phase and Condition 1 in this example was the same as Experiment 4 in Example 1. An increase in temperature to 35° C. and a manual cell bleed of 5% were used in Condition 2 to test the effect on cell culture performance. In the third condition, the manual bleed was stopped and the effects on culture observed.

The culture was maintained until a permittivity target of 115 pF/cm was achieved (Day 6), Table 8. This permittivity was chosen to achieve a high VCD (>1E8 cells/mL) in the biomass intensification phase. The minimal packed cell volume was ≥25%. The biomass-specific permeate rate was 0.0157 pF/cm·day. No manual cell bleed was used to maintain the desired permittivity. The biomass-specific permittivity was 0.0157 pF/cm·day.
Condition 1: The culture temperature was lowered to 34.0° C. and the permittivity was lowered to 105 pF/cm. The decrease in permittivity was made to help lower the biomass in culture and reduce the risk ATF failure. The permeate rate was maintained at 18 wv/day until day 14. No manual cell bleed was used to achieve or maintain the desired permittivity. The biomass-specific permittivity was 0.0171 pF/cm·day.
Condition 2: The culture temperature was increased to 35.0° C. The permittivity was maintained at 105 pF/cm by a manual cell bleed of 5%. The permeate rate was maintained at 18 wv/day until day 20. The biomass-specific permittivity was 0.0171 pF/cm·day.
Condition 3: The culture temperature permittivity and permeate rate were all maintained as in Condition 2, but no manual cell bleed was used. The biomass-specific permittivity was 0.0171 pF/cm·day.

During Conditions 1-3 the recombinant product in the permeate flow was continuously harvested into sterile harvest bags. The culture was terminated on Day 28.

### Example 2 Results

Condition 1 resulted in non-steady state CM as expected. This was characterized by declining VCD, viability and high protein production rates.

The bleed in Condition 2 served to remove a portion of the cells and cell debris in the culture, and as such VCD declined in Condition 2, despite the higher temperature. This cell line increased PCV (and cell diameter) very dramatically, as compared to the cell line in Example 1. From experience, it was known that a PCV of 30-35% increased the risk of ATF failure in the setup being used. The cell bleed was required in Condition 2, to help maintain or lower the PCV, lower the risk of ATF failure and prolong the cell culture duration.

As a result of the increase in temperature since Day 15 and the lack of manual cell bleed in Condition 3 starting Day 21, there was a gradual and sustained increase in VCD and no decline in viability. This condition demonstrated that an increase in temperature would transition the culture away from an aggressive non-steady state culture favoring protein production towards a steady state culture with cell growth. The protein production rate in Condition 3 was lower than Condition 1 as expected due to the increase in temperature that favored cell growth. The PQ was similar in the two lots of this CM run (see Table 9). This demonstrates that the process is in a state of control and that the process conditions tested are suitable for cell culture.

The growth, biomass intensification and Condition 1 in this Example 2 are the same as in Experiment 4 in Example 1. Similar to Example 1, the VCD and viability dropped and PCV increased in Example 2. The rates of change in viability, VCD and PCV were different than the rates of change observed in Example 1 since the different cell lines/molecules have different magnitude of responses to process setpoints. The production rate was very similar between the two bispecific T cell engagers. In Example 2, the viability dropped and the PCV increased than in Example 1, Experiment 4 due to differences in the cell line and bispecific T cell engager that was used.

TABLE 9
Product quality attributes of two lots
of continuous perfusion bioreactor

CM Process

| Attribute | Lot 1 | Lot 2 |
|---|---|---|
| PQA (%) | (D 7-17) | (D 18-28) |
| SEC HMW | 19.48 | 17.53 |
| CEX Basic | 30.23 | 30.48 |
| CEX Acidic | 5.67 | 6.46 |
| rCE LMW | 1.68 | 2.76 |

The product quality from both lots in the CM run were similar, which demonstrates that the process is in a state of control.

### Example 2 Conclusion

In this experiment non-steady state CM, along with a biomass intensification phase, has been demonstrated in a different cell line, producing a different molecule. The transition to a steady state CM operation is also demonstrated. This shows that this cell line also demonstrates distinctive cell culture performance characteristics in these two process design spaces, i.e., favoring protein production over cell growth in the non-steady state space and favoring cell growth over protein production in the steady state CM space. The process can be modulated from one type of operation to another by changing the process temperature.

### Example 3

Example 3 illustrates non-steady state cell culture on a third, different cell line and antigen-binding protein, which demonstrates repeatability of these methods. The cell density and permittivity in this example were lower than the first two examples.

A 50 L single use bioreactor (Hyclone) was inoculated with CHO cells expressing a second bispecific T cell engager at a density of 38×10⁵ cells/ml in 25 L of serum-free, chemically defined basal medium. Following inoculation, the target volume in the bioreactor was adjusted to 45 L of basal medium, at a setpoint pH of 6.90 and an initial temperature of 36.0° C. and maintained for 144 hours.

The bioreactor was equipped with one ATF 6 (0.2p) alternating tangential flow devices (Refine, Pine Brook, NJ). On Day 1, the culture was continuously perfused with a serum-free, chemically defined perfusion medium. The permeate flow rate was adjusted daily to reach a maximal working volume (WV) per day of 1.8 on Day 6, see Table 10. Process parameters for the experiment are shown in Table 10. The cells remained in the retenate and returned to the bioreactor to build and/or maintain biomass. Any recombinant product passed in the permeate flow and was discharged to waste during the growth phase (Days 0-6).

Cell count, viability, cell diameter, pH, pCO₂, pO₂, permittivity, glucose, lactate, ammonia, osmolality, packed cell volume, bioreactor titer, permeate titer, and harvest titer were measured daily. Bulk bioreactor supernatant and harvest samples were taken for PQ.

TABLE 10
Permeate flowrate schedule

| Day | WV per Day |
|---|---|
| 2 | 0.4 |
| 3 | 0.5 |
| 4 | 0.75 |
| 5 | 1.0 |
| 6 | 1.8 |

TABLE 11
Process parameter setpoints

| | Time | Temperature | Permittivity | Permeate Rate |
|---|---|---|---|---|
| Phase | (days) | (° C.) | (pF/cm) | (wv/day) |
| Growth | 0-6 | 36 | 70 | 0-1.8 |
| Condition 1 | 7-8 | 34 | 60 | 1.8 |
| Condition 2 | 9-15 | 34 | 60 | 1.8 |
| Condition 3 | 16-17 | 36 | 60 | 1.8 |

The culture was maintained until a permittivity target of 70 pF/cm was achieved (Day 6).
Example 3 Condition 1: The culture temperature was lowered to 34.0° C. to control cell growth and promote protein production. Permittivity decreased to 70 pF/cm and the permeate rate was maintained at 1.8 wv/day until Day 15.
Example 3 Condition 2. The culture temperature was increased to 34.0° C., the permittivity and permeate rate was decreased to 60 pF/cm. During both Condition 1 and 2, the recombinant product in the permeate flow was continuously harvested into sterile harvest bags. The culture was continued under these conditions until the culture was terminated.
Condition 3. The culture temperature was increased to 36.0° C., the permittivity and permeate rate were maintained as in Condition 1. During both Condition 1 and 2, the recombinant product in the permeate flow was continuously harvested into sterile harvest bags. The culture was continued under these conditions until the culture was terminated

### Example 3 Results

The experiment demonstrated that biomass intensification and non-steady state starting early in the cell culture with low temperature, resulted in a high protein production rate. Decreasing the permittivity in Condition 2, further increased protein production by while viability was declining. Increasing the temperature and permittivity in Condition 3, allowed for the culture to maintain viability.

### Example 3 Conclusion

In conclusion, permittivity and temperature are levers that can be utilized to increase protein production.

### Summary of Process Parameters in the Examples

Tables 12 and 13 below summarize exemplary process parameters values contemplated herein as useful for improved protein production.

TABLE 12
Average and Ranges of process parameter values

| | Day 0 | Day 7 to | Day 18 to end |
|---|---|---|---|
| Process Parameter | to 6 | Day 17 | of culture |
| VCD (10⁶ cells/mL) | 38 | 75 | 39 |
| | (4 to 106) | (41 to 129) | (28 to 66) |
| Bleed (%) | 1.5 | 14 | 1 |
| | (0 to 3) | (0 to 35) | (0 to 17) |
| Viability (%) | 97 | 88 | 53 |
| | (94 to 99) | (60 to 98) | (38 to 89) |
| PCV (%) | 10 | 28 | 35 |
| | (2 to 24) | (22 to 40) | (27 to 45) |

TABLE 13
Average and Ranges of daily change in process parameters

| Process Parameter | Day 0 | Day 7 to | Day 18 to end |
|---|---|---|---|
| Daily Change | to 6 | Day 17 | of culture |
| VCD Change (%/day) | 60 | −5 | −3 |
| | (40to 107) | (−2 to −25) | |
| Bleed Change (%/day) | N/A | −11, | N/A |
| | | (0 to −51) | |
| Viability Change (%/day) | 0 | −3 | −5 |
| | (−3 to 1.6) | (−0.5 to −10) | |
| PCV Change (%/day) | 57 | 3.7 | −2.5 |
| | (40 to 89) | (0 to 5) | |

All documents referred to in this application are hereby incorporated by reference in their entirety.

We claim:

1. A method of producing a protein product in a bioreactor in continuous perfusion mode, the method comprising a growth phase followed by a production phase which is not operated at steady state cell culture conditions,
wherein the growth phase comprises the steps of:
(a) inoculating cells expressing the protein product and liquid media into a bioreactor at a high cell density, and
(b) growing the cells at a set temperature and progressively higher permeate rates to a first biomass setpoint; and
wherein the non-steady state production phase comprises the steps of:
(c) shifting to a lower temperature or a lower permeate rate when the first biomass setpoint is reached to begin to transition the culture towards protein production,
(d) growing the cells at a set temperature and permeate rate up to a second higher biomass setpoint that promotes non-steady state cell culture and high productivity,
(e) culturing the cells at the stated culture conditions in (d), such that the viability decreases over time, and
(f) collecting the protein product from a harvest stream during the production phase.

2. The method of claim 1, wherein the variable cell density (VCD) declines over time or the packed cell volume (PCV) increases over time.

3. The method of claim 1, wherein one or more cell bleeds are performed so that the culture does not exceed the biomass setpoints.

4. The method of claim 3, wherein a bleed occurs once the cells increase past the second biomass setpoint and the bleed rate decreases or falls to zero.

5. The method of any preceding claim, wherein the high cell density of (a) is about 0.2 million cells/mL to about 5 million cells/mL.

6. The method of any preceding claim, wherein the first biomass setpoint is about 50 million cells/mL to about 100 million cells/mL.

7. The method of any preceding claim, wherein the second biomass setpoint is about 100 million cells/mL to about 150 million cells/mL.

8. The method of any preceding claim, wherein the permeate rate of (b) is 0 to about 4.1 working volumes/day.

9. The method of any preceding claim wherein the permeate rate of (d) is about 1.0 to about 4.2 working volumes/day.

10. The method of claim 2 wherein the VCD maximum of (d) is about 130 million to about 140 million cells/mL.

11. The method of any preceding claim wherein the set temperature of (b) is about 35.5 to about 36.5° C.

12. The method of any preceding claim wherein the temperature shift of (c) is to a temperature of about 32.5 to about 35.5° C.

13. The method of any preceding claim wherein the cell bleed in the growth phase is from about 0% to about 3%.

14. The method of any preceding claim wherein the cell viability in the growth phase is about 90% to about 99%.

15. The method of claim 14, wherein the cell viability in step (e) decreases to a viability of about 30% to about 80%.

16. The method of any preceding claim, wherein the PCV in the growth phase increases to about 2% to about 24%.

17. The method of claim 16, wherein the PCV in the production phase is about 25% to about 50%.

18. The method of any preceding claim, wherein the duration of the growth phase is about four to twelve days.

19. The method of any preceding claim, wherein the duration of the production phase before cell growth stops is about ten days.

20. The method of any preceding claim, wherein the duration of the production phase after cell growth stops is about nine to about forty-one days.

21. The method of any preceding claim further comprising subjecting the harvested protein product to downstream steps of capture chromatography, viral inactivation and/or polishing steps.

22. The method of any preceding claim, wherein the cells are mammalian cells.

23. The method of any preceding claim, wherein the mammalian cells are Chinese hamster ovary (CHO) cells.

24. The method of any preceding claim, wherein the protein product is an antigen-binding protein.

25. The method of claim 24, wherein the antigen-binding protein is an antibody, an antibody fragment, an antibody derivative, an antibody analog, an antibody construct, a fusion protein, a mutein, a multispecific protein, a bispecific protein, a bispecific T-cell engager, or a peptibody.
